(19) United States
(12) Patent Application Publication
(10) Pub. No.: US 2005/0042763 A1
(43) Pub. Date: Feb. 24, 2005

(54) Testing using diesel exhaust produced by a non-engine based test system

(75) Inventors: Andy Morris Anderson, San Antonio, TX (US); Cynthia Chaffin Webb, San Antonio, TX (US); Rijing Zhan, San Antonio, TX (US); Gordon James Johnston Bartley, San Antonio, TX (US)

Correspondence Address: PAULA D. MORRIS & ASSOCIATES, P.C.;d/b/a THE MORRIS LAW FIRM, P.C., 10260 WESTHEIMER, SUITE 360, HOUSTON, TX, 77042-3110, US

(73) Assignee: Southwest Research Institute, San Antonio, TX (US)

(21) Appl. No.: 10/917,245

(22) Filed: Aug. 12, 2004

Related U.S. Application Data
(63) Continuation-in-part of application No. 10/213,890, filed on Aug. 6, 2002.

(51) Int. Cl. G01N 33/00

(52) U.S. Cl. 436/137

(57) ABSTRACT

Method for treating a component using a non-engine based test system to produce and/or to regenerate a contaminated component comprising contaminant particulates from diesel exhaust.

### PRIORITY DATA

The present application is a continuation-in-part of U.S. patent application Ser. No. 10/213,890, filed Aug. 6, 2002, published May 1, 2003 as US 2003-0079520 b 1; (pending), incorporated herein by reference.

### RELATED APPLICATIONS

The present application also is related to application Ser. No. 10/457,916, published Jan. 15, 2004 as US 2004-0007056 A1 (pending); application Ser. No. 10/439,146, published Feb. 12, 2004 as US 2004-0025580 A1 (pending); application Ser. No. 10/458,023, published Feb. 12, 2004 as US 2004-0028588 A1 (pending); and, application Ser. No. 10/847,034, filed May 17, 204 (pending).

### FIELD OF THE INVENTION

The present application relates to methods using a non-engine based test system to produce diesel exhaust for testing and/or regenerating diesel aftertreatment components.

### BACKGROUND

Diesel powered engines are used to conduct a variety of tests on diesel engine aftertreatment devices, including aging and regeneration testing of diesel particulate filters. It is desirable to conduct such tests with a high level of precision at the lowest cost possible. Historically, the use of diesel engines to perform the above procedures has presented many disadvantages including inconsistent operability, intensive maintenance, and expensive operating costs. Methods are needed which overcome the foregoing deficiencies.

### SUMMARY OF THE INVENTION

The present application provides a method for testing a component. The method comprises: providing a non-engine based test system comprising a combustor in fluid communication with the component; supplying diesel fuel and air to the combustor at a controlled air to fuel ratio (AFR) and under feed conditions effective to produce a feedstream flowpath effective to prevent substantial damage to the combustor; combusting at least a portion of the diesel fuel in the feed stream flowpath under combustion conditions effective to produce diesel exhaust comprising one or more particulates; and, exposing the component to the diesel exhaust under test conditions effective to produce a contaminated component comprising an amount of diesel contaminant particulates.

### BRIEF DESCRIPTION OF THE FIGURES

FIG. 1 is a schematic diagram of the FOCAS® rig, a preferred non-engine based exhaust component rapid aging system for use herein, described in U.S. patent application Publication No. 20030079520, published May 1, 2003, incorporated herein by reference.

### BRIEF DESCRIPTION

Numerous test methods have been used in the past to simulate aging and regeneration of diesel aftertreatment components. Diesel exhaust comprises particulates, which are believed to be unburned carbon particulates or “soot.” The particulates generally are removed from the exhaust using a diesel particulate filter.

In the past, a diesel particulate filter was aged by exposing the diesel particulate filter to diesel exhaust created by a diesel powered engine, thereby loading the filter with particulates from the diesel exhaust. The diesel powered engine generally was on a bench stand in the laboratory or on a motor vehicle. Examples of diesel powered engines used in such tests include but are not necessarily limited to the Cummins ISM (10.8 liter), the Navistar T444E (7.3 liter V-8), and the Detroit Diesel Series 60 PSA DW-12 (2.2L). The diesel powered engine had to be run at various high speed and load conditions cyclically for several hours in order to simulate aging of the diesel particulate filter. Higher temperatures could be generated using a diesel powered engine only by post injection or in-exhaust injection of fuel, rendering generation of temperatures up to 650° C. relatively expensive and fuel inefficient.

The present application substitutes a non-engine based exhaust component rapid aging system (NEBECRAS) for a diesel powered engine in such tests to produce diesel exhaust gas. The use of a NEBECRAS in place of a diesel powered engine lowers operating costs, reduces test variability, and increases control of the exhaust gas composition, pressure, mass flow rate, and temperature. The NEBECRAS also can be used to regenerate the diesel particulate filter.

Non-Engine Based Exhaust Component Rapid Aging System

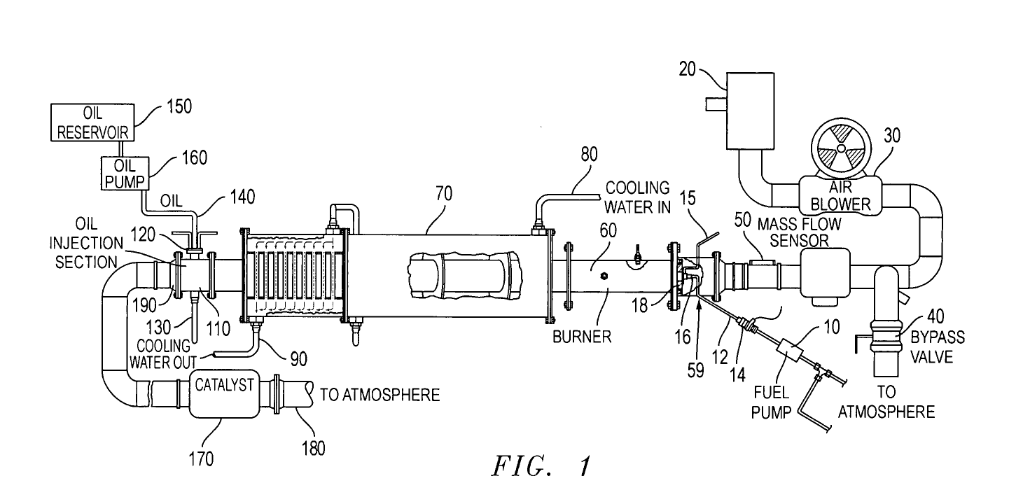

U.S. patent application Publication No. 20030079520 describes a preferred NEBECRAS (the FOCAS® rig) for use in the present method(s). Briefly, the FOCAS® rig comprises: (1) an air supply system to provide air for combustion to the burner, (2) a fuel system to provide fuel to the burner, (3) a burner system to combust the air and fuel mixture and to provide the proper exhaust gas constituents, (4) a heat exchanger to control the exhaust gas temperature, (5) (optionally) an oil injection system, and (6) a computerized control system. The foregoing components are illustrated in FIG. 1 and described in detail in U.S. patent application Publication No. 20030079520, which has been incorporated herein by reference and will not be described in detail herein.

The FOCAS® rig was developed to evaluate the long term effects of individual variables on the long term performance of exhaust aftertreatment devices (i.e., a catalyst). The FOCAS® rig produces exhaust gas with a composition and temperature corresponding to that produced by the internal combustion engine of a motor vehicle.

The burner system in the FOCAS® rig is effective to substantially stoichiometrically combust at least a portion of fuel in the feedstream flowpath without substantial damage to the combustor. In a preferred embodiment, the combustor comprises a nozzle comprising a swirl plate which is effective even at a stoichiometric air to fuel ratio (AFR) of producing a feedstream flowpath comprising an air shroud effective to prevent flame from attaching to a nozzle supplying fuel and air to the combustor during combustion of fuel. The feedstream flowpath also preferably prevents flame from remaining in constant contact with an inner wall of the combustor during combustion of fuel.

Although the FOCAS® rig is a preferred NEBECRAS, it will be apparent to persons of ordinary skill in the art that any functional and effective non-engine based test system could be adapted for use in accordance with the principles described herein.

The NEBECRAS may be used to perform substantially any test which requires diesel exhaust and to test substantially any diesel engine exhaust component, preferably diesel engine exhaust aftertreatment comoponents. The NEBECRAS also may be used to regenerate substantially any particulate contaminated component. Preferred exhaust components for treatment and/or regeneration by the NEBECRAS include but are not necessarily limited to catalyzed and non-catalyzed diesel particulate filters (DPFs), lean NOₓ catalysts (LNTs), and diesel oxidation catalysts (DOCs).

FIG. 1 illustrates the FOCAS® rig (a preferred NEBECRAS). The FOCAS® rig includes a standard automotive fuel pump 10 capable of pumping diesel fuel through the fuel line 12l to an electronically actuated fuel control valve 14 then to the burner 60 producing continuous stoichiometric combustion of fuel. Under typical test conditions, the FOCAS® rig is programmable to produce diesel exhaust that is passed directly to the diesel particulate filter. If desired, the FOCAS® rig provides substantially continuous and effective stoichiometric combustion for 200 hours or more without the need for maintenance. In a preferred embodiment, the burner may run substantially continuously at stoichiometric for at least 1500 hours with minimal maintenance.

The burner system in the FOCAS® rig may be used to generate stoichiometric, rich, and lean hot gas conditions without substantial damage to the combustor. In a preferred embodiment, the combustor of the FOCAS® rig comprises a feed member comprising a swirl plate which is effective even at a stoichiometric air to fuel ratio (AFR) of producing a feedstream flowpath comprising an air shroud effective to prevent flame from attaching to a feed member during combustion of fuel. The feedstream flowpath also preferably prevents flame from remaining in constant contact with an inner wall of the combustor during combustion of fuel.

The FOCAS® rig may be programmed to produce diesel exhaust or alternative exhaust compositions with increased or decreased amounts of a particular exhaust component or components, for example, to simulate diesel exhaust produced in cold climates, at high altitudes, during engine acceleration conditions (i.e., increased NOx production), during engine deceleration conditions (i.e., decreased NOx production), and combinations thereof. For example, an exhaust gas can be programmed to achieve fuel sulfur levels including but not necessarily limited to 15-ppm, 30-ppm, 50-ppm, 100-ppm, and 300-ppm, etc., depending on the desired particulate matter requirements of the exhaust gas.

A catalytic converter may be positioned with respect to the diesel particulate filter to assist in simulating a preferred diesel exhaust. For instance, the FOCAS® rig may include a NOₓ reducing catalyst upstream (or downstream) of the diesel particulate filter simulating a NOₓ reduced exhaust gas product contacting the filter. In the alternative, the FOCAS® rig may simply be programmed to simulate a NOₓ reduced exhaust gas without using a reducing catalyst.

The FOCAS® rig may be deactivated, the system may be cooled to ambient conditions in a matter of minutes, and then immediately after cooling (if desired), the system can be used to perform additional testing. The FOCAS® rig offers improved repeatability and reduced cool down time. The FOCAS® rig also offers relatively easy maintenance compared to diesel engines, which require periodic maintenance (oil changes, tune-ups) and time consuming repairs. The FOCAS® rig is relatively simple (with less moving parts and friction areas) and can operate with improved fuel economy when operated lean. These advantages make it highly desirable as a research and development tool.

Diesel Exhaust

Diesel fuel generally boils in a range of from about 140° C. to about 400° C., typically from about 150° C. to about 370° C. As explained above, diesel fuel generally produces a diesel exhaust comprising particulates. The accuracy of a given simulation using diesel exhaust depends, at least in part, on the exhaust gas composition.

The main components of diesel exhaust include but are not necessarily limited to carbon monoxide (CO), carbon dioxide (CO₂), oxides of nitrogen (NOₓ), oxides of sulfur; hydrocarbons (HC); unburned carbon particulate matter; oxygen (O₂); and/or nitrogen (N₂). Other compounds also may be included in the exhaust gas depending on the desired test conditions. Preferred compounds comprise components selected from the group consisting of phosphorous, zinc, sulfur and combinations thereof. The quantity and composition of diesel exhaust during testing using diesel powered engines varies depending on a number of factors, including but not necessarily limited to: wear and tear on engine moving parts; quality of lubrication oil; uncontrolled lubrication oil consumption; quality of the diesel fuel; type of engine; engine tuning; fuel pump setting; the workload demand on the engine; engine temperature; and, engine maintenance.

Using a NEBECRAS in place of a diesel engine minimizes or eliminates the above factors, allowing more control over the quantity and composition of the diesel exhaust.

Under normal diesel engine operating conditions, carbon monoxide (CO), hydrocarbons (HC), and aldehydes are generated in the exhaust as the result of incomplete combustion of fuel. A significant portion of exhaust hydrocarbon also is derived from the engine lube oil. Nitrogen oxides (NOₓ) are generated from nitrogen and oxygen under the high pressure and temperature conditions in the engine cylinder. NOₓ consists mostly of nitric oxide (NO) and a small fraction of nitrogen dioxide (NO₂). Sulfur dioxide (SO₂) is generated from the sulfur present in diesel fuel and lubricant oil, and the concentration of SO₂ in the exhaust gas depends on the sulfur content of the fuel and the lubricant oil. The FOCAS® rig is designed to burn any number of fuels to reproduce an exhaust product comprising any combination of the above listed components.

One of the main components of diesel exhaust is particulates, which generally comprise unburned carbon particulate matter comprising aggregates of dry particulates and wet particulates. The dry particulates generally comprise carbon, metal, adsorbed organic compounds (i.e., hydrocarbons), and varying amounts of sulfates, nitrates, and combinations thereof. Examples of adsorbed organic compounds are aldehydes, polycyclic aromatic hydrocarbons (also called PAH's), and combinations thereof. Unburned hydrocarbons, which generate a characteristic diesel odor, include but are not necessarily limited to aldehydes, such as formaldehyde, acrolein, and combinations thereof. Like carbon monoxide, the aldehydes are the product of incomplete combustion. The amount of sulfates in the diesel exhaust is directly related to the sulfur content of the diesel fuel. The dry particulates also may comprise trace elements, including but not necessarily limited to zinc, phosphorus, and cesium. Wet particulates generally comprise up to about 60 wt. % of the soluble organic fraction (hydrocarbons adsorbed and condensed on carbon particles), while the dry particulates are comprised mostly of dry carbon.

Diesel particulates are very fine. The primary (nuclei) carbon particles have an average diameter from about 0.01 microns to about 0.08 microns, while the aggregates have an average diameter from about 0.08 microns to about 1 microns. The actual composition of the diesel particulates depends, to some degree, on the particular engine used to produce the particulates and on its load and speed conditions. The actual composition of the particulates also depends on the thermodynamic conditions in the diesel exhaust and the particulate collection system being used. For example, under normal engine operating conditions, particles become coated with adsorbed and condensed high molecular weight organic compounds. These include unburned hydrocarbons, oxygenated hydrocarbons (ketones, esters, organic acids) and polynuclear aromatic hydrocarbons (PAH's).

The NEBECRAS is programmed to generate a diesel exhaust comprising a desired composition and average diameter of diesel particulates. Diesel exhaust produced at near stoichiometric conditions typically comprises the following components in the ranges given below:

| | | Particulate | | |
|---|---|---|---|---|
| CO | HC | Matter | NO_{X} | SO₂ |
| ppm | ppm | g/m3 | ppm | ppm |
| 5-1,500 | 20-400 | 0.1-0.25 | 50-2,500 | 0-150 |

Diesel Particulate Filter

A diesel particulate filter is any apparatus that collects and retains particulates from a diesel exhaust contacting the filter. In general, a diesel particulate filter consists of a porous substrate or ceramic fiber that traps the particulates but permits gases in the diesel exhaust to pass through. A suitable diesel particulate filter reduces the content of particulates in a diesel exhaust by about 50% or more in terms of grams per horsepower per hour (g/hp-hr), preferably by about 50% to about 90% g/hp-hr or more. A suitable diesel particulate filter traps particulates having an average diameter of about 100 nm or less.

Regarding regeneration methods, there are two basic types of diesel particulate filters, passive and active. Most passive diesel particulate filters remove particulates by collecting particles in the filter and oxidizing them during vehicle use, preferably substantially continuously. Active diesel particulate filters typically comprise one or more catalyst(s) effective to catalyze the oxidation of the particulates and/or aggregates at common engine exhaust temperatures.

Aging of Diesel Particulate Filters

Generally, diesel particulate filter aging is evidenced by a reduction in filtration efficiency. The reduction in filtration efficiency generally is due to multiple soot binding/regeneration cycles, which may lead to ash buildup in the diesel particulate filter. Aging also depends on numbers factors including but not necessarily limited to exposure time of the diesel particulate filter to the exhaust gas, the flowrate of the exhaust gas through the filter, the amount of pressure drop during use, the porosity of the filter, the filter materials used, and ambient humidity.

During aging of a diesel particulate filter, the flowrate generally is maintained at from about 0 to about 300 standard cubic feet per minute (scfm) and the exhaust temperature generally is maintained at from about 150° C. to about 650° C., typically from about 150° C. to about 300° C., for about 1 hour or more, and up to about 250 hours or more. Exposure times typically are determined according to the type of diesel particulate filter being tested, the components of the exhaust gas product, the desired aging conditions to be simulated, and combinations thereof.

Once desired aging requirements have been met, the diesel particulate filter is evaluated and/or regenerated.

Regeneration of the Diesel Particulate Filter

Regeneration of the diesel particulate filter involves removal of the particulate matter from the filter such that the final diesel exhaust, or the exhaust produced by passing the diesel exhaust through the diesel particulate filter, meets applicable EPA guidelines.

Generally, the exhaust gas temperature is increased to a temperature sufficiently high to autoignite and sustain combustion of the particulate matter on the filter. Regeneration temperatures generally are from about 300° C. to about 650° C., preferably about 350° C. or higher for catalyzed diesel particulate filter depending on catalyst formulation, and about 600° C. or higher for most uncatalyzed diesel particulate filters. The contaminant particulates generally must attain a minimum temperature of about 500° C. to about 650° C., preferably from about 550° C. to about 650° C., most preferably from about 585° C. to about 625° C. to autoignite and sustain combustion. The desired temperature is determined by factors including, but not necessarily limited to the type of diesel particulate filter being tested, the fuel sulfur levels, NOₓ levels, O₂ levels, soot levels, and combinations thereof. The NEBECRAS produces diesel exhaust at temperatures of up to about 650° C., which is sufficiently high to desulfurize a component.

For rapid oxidation of the particulate matter, there must be sufficient free oxygen available, preferably from about 3 vol. % to 20 vol. % of the exhaust stream. The temperature and oxygen levels are maintained until the regeneration is complete, which typically requires about 20 minutes or less. During typical vehicle operation, active regeneration of the diesel particulate filter is needed regularly, depending on the engine, exhaust catalyst, and diesel particulate filter performance. The NEBECRAS may be programmed to reproduce a desired aging cycle and/or a desired regeneration cycle, alone or in combination, once or multiple times, as desired.

The regeneration preferably is effective to remove or oxidize a sufficient quantity of particulate matter from the diesel particulate filter to meet EPA Tier 1 Emission Standards for Passenger Cars and Light-Duty Trucks, FTP 75, g/mi:

TABLE 1
EPA Tier 1 Emission Standards for Passenger Cars and Light-Duty Trucks, FTP 75, g/mi

| | 50,000 miles/5 yrs | | | | | | 100,000 miles/10 years¹ | | | | | |
|---|---|---|---|---|---|---|---|---|---|---|---|---|
| | | | | NOx | NOx | | | | | NOx | NOx | |
| Category | THC | NMHC | CO | diesel | gasoline | PM | THC | NMHC | CO | diesel | gasoline | PM |
| Passenger cars | 0.41 | 0.25 | 3.4 | 1.0 | 0.4 | 0.08 | — | 0.31 | 4.2 | 1.25 | 0.6 | 0.10 |
| LLDT, LVW | — | 0.25 | 3.4 | 1.0 | 0.4 | 0.08 | 0.80 | 0.31 | 4.2 | 1.25 | 0.6 | 0.10 |
| <3,750 lbs | | | | | | | | | | | | |
| LLDT, LVW | — | 0.32 | 4.4 | — | 0.7 | 0.08 | 0.80 | 0.40 | 5.5 | 0.97 | 0.97 | 0.10 |
| >3,750 lbs | | | | | | | | | | | | |
| HLDT, ALVW | 0.32 | — | 4.4 | — | 0.7 | — | 0.80 | 0.46 | 6.4 | 0.98 | 0.98 | 0.10 |
| <5,750 lbs | | | | | | | | | | | | |
| HLDT, ALVW | 0.39 | — | 5.0 | — | 1.1 | — | 0.80 | 0.56 | 7.3 | 1.53 | 1.53 | 0.12 |
| >5,750 lbs | | | | | | | | | | | | |

¹Useful life 120,000 miles/11 years for all HLDT standards and for THC standards for LDT (light duty truck)
Abbreviations:
THC-total hydrocarbons
NMHC-non-methane hydrocarbons
PM-particulate matter
LVW-loaded vehicle weight (curb weight + 300 lbs)
ALVW-adjusted LVW (the numerical average of the curb weight and the GVWR)
LLDT-light light-duty truck (below 6,000 lbs GVWR)
HLDT-heavy light-duty truck (above 6,000 lbs GVWR)

Preferably, the regeneration is effective to remove or oxidize a sufficient quantity of particulate matter to meet Tier 2 Emission Standards FTP 75, g/mi:

TABLE 2
Tier 2 Emission Standards, FTP 75, g/mi

| | 50,000 miles | | | | | 120,000 miles | | | | |
|---|---|---|---|---|---|---|---|---|---|---|
| Bin# | NMOG | CO | NOx | PM | HCHO | NMOG | CO | NOx* | PM | HCHO |

Temporary Bins

| | | | | | | | | | | |
|---|---|---|---|---|---|---|---|---|---|---|
| MDPV^{c} | | | | | | 0.280 | 7.3 | 0.9 | 0.12 | 0.032 |
| 10^{a,b,d,f} | 0.125 | 3.4 | 0.4 | — | 0.015 | 0.156 | 4.2 | 0.6 | 0.08 | 0.018 |
| | (0.160) | (4.4) | | | (0.018) | (0.230) | (6.4) | | | (0.027) |
| 9^{a,b,e} | 0.075 | 3.4 | 0.2 | — | 0.015 | 0.090 | 4.2 | 0.3 | 0.06 | 0.018 |
| | (0.140) | | | | | (0.180) | | | | |

Permanent Bins

| | | | | | | | | | | |
|---|---|---|---|---|---|---|---|---|---|---|
| 8^{b} | 0.100 | 3.4 | 0.14 | — | 0.015 | 0.125 | 4.2 | 0.20 | 0.02 | 0.018 |
| | (0.125) | | | | | (0.156) | | | | |
| 7 | 0.075 | 3.4 | 0.11 | — | 0.015 | 0.090 | 4.2 | 0.15 | 0.02 | 0.018 |
| 6 | 0.075 | 3.4 | 0.08 | — | 0.015 | 0.090 | 4.2 | 0.10 | 0.01 | 0.018 |
| 5 | 0.075 | 3.4 | 0.05 | — | 0.015 | 0.090 | 4.2 | 0.07 | 0.01 | 0.018 |
| 4 | — | — | — | — | — | 0.070 | 2.1 | 0.04 | 0.01 | 0.011 |
| 3 | — | — | — | — | — | 0.055 | 2.1 | 0.03 | 0.01 | 0.011 |
| 2 | — | — | — | — | — | 0.010 | 2.1 | 0.02 | 0.01 | 0.004 |
| 1 | — | — | — | — | — | 0.000 | 0.0 | 0.00 | 0.00 | 0.000 |

*average manufacturer fleet NOx standard is 0.07 g/mi
Abbreviations:
NMOG-non-methane organic gases
HCHO-formaldehyde
^{a}Bin deleted at end of 2006 model year (2008 for HLDTs)
^{b}The higher temporary NMOG, CO and HCHO values apply only to HLDTs and expire after 2008
^{c}An additional temporary bin restricted to MDPVs, expires after model year 2008
^{d}Optional temporary NMOG standard of 0.195 g/mi (50,000) and 0.280 g/mi (120,000) applies for qualifying LDT4s and MDPVs only
^{e}Optional temporary NMOG standard of 0.100 g/mi (50,000) and 0.130 g/mi (120,000) applies for qualifying LDT2s only
^{f}50,000 mile standard optional for diesels certified to bin 10

Persons of ordinary skill in the art will recognize that many modifications may be made to the present application without departing from the spirit and scope of the application. The embodiment described herein is meant to be illustrative only and should not be taken as limiting the application, which is defined in the claims.

1. A method for testing a component, the method comprising:
providing a non-engine based test system comprising a combustor in fluid communication with the component;
supplying diesel fuel and air to the combustor at a controlled air to fuel ratio (AFR) and under feed conditions effective to produce a feedstream flowpath effective to prevent substantial damage to the combustor;
combusting at least a portion of the diesel fuel in the feedstream flowpath under combustion conditions effective to produce diesel exhaust comprising one or more particulates; and
exposing the component to the diesel exhaust under test conditions effective to produce one or more contaminated components comprising an amount of diesel contaminant particulates.

2. The method of claim 1 wherein the test conditions comprise aging conditions.

3. The method of claim 1 wherein the one or more contaminated components are selected from the group consisting of catalyzed and non-catalyzed diesel particulate filters (DPFs), lean NOₓ catalysts (LNTs), and diesel oxidation catalyst (DOCs).

4. The method of claim 2 wherein the one or more contaminated components are selected from the group consisting of catalyzed and non-catalyzed diesel particulate filters (DPFs), lean NOₓ catalysts (LNTs), and diesel oxidation catalyst (DOCs).

5. The method of claim 1 further comprising exposing the one or more contaminated components to regeneration conditions effective to reduce the amount of diesel contaminant particulates, producing one or more regenerated components

6. The method of claim 2 further comprising exposing the one or more contaminated components to regeneration conditions effective to reduce the amount of diesel contaminant particulates, producing one or more regenerated components.

7. The method of claim 3 further comprising exposing the one or more contaminated components to regeneration conditions effective to reduce the amount of diesel contaminant particulates, producing one or more regenerated components

8. The method of claim 4 further comprising exposing the one or more contaminated components to regeneration conditions effective to reduce the amount of diesel contaminant particulates, producing one or more regenerated components.

9. The method of claim 6 further comprising
providing a test diesel exhaust comprising a first quantity of particulate matter in grams per horsepower per hour (g/hp-hr); and,
passing the test diesel exhaust through one of the regenerated diesel particulate filters to produce a resulting final diesel exhaust comprising a second quantity of particulate matter which is about 50% or less (g/hp-hr) than the first quantity of particulate matter.

10. The method of claim 8 further comprising
providing a test diesel exhaust comprising a first quantity of particulate matter in grams per horsepower per hour (g/hp-hr); and,
passing the test diesel exhaust through one of the regenerated diesel particulate filters to produce a resulting final diesel exhaust comprising a second quantity of particulate matter which is from about 50% to about 90% (g/hp-hr) less than the first quantity of particulate matter.

11. The method of claim 1 wherein the combustion conditions are effective to produce diesel exhaust comprising exhaust components selected from the group consisting of carbon monoxide, carbon dioxide, oxides of nitrogen, oxides of sulfur, hydrocarbons, unburned carbon particulate matter, oxygen, nitrogen, and combinations thereof.

12. The method of claim 8 wherein the combustion conditions are effective to produce diesel exhaust comprising exhaust components selected from the group consisting of carbon monoxide, carbon dioxide, oxides of nitrogen, oxides of sulfur, hydrocarbons, unburned carbon particulate matter, oxygen, nitrogen, and combinations thereof.

13. The method of claim 10 wherein the combustion conditions are effective to produce diesel exhaust comprising exhaust components selected from the group consisting of carbon monoxide, carbon dioxide, oxides of nitrogen, oxides of sulfur, hydrocarbons, unburned carbon particulate matter, oxygen, nitrogen, and combinations thereof.

14. The method of claim 13 wherein the combustion conditions are effective to produce diesel exhaust further comprising exhaust components selected from the group consisting of phosphorous, zinc, aldehydes, nitrogen dioxide, sulfur dioxide.

15. The method of claim 12 wherein the combustion conditions are effective to produce diesel exhaust comprising:
from about 5 to about 1500 ppm carbon monoxide;
from about 20 to about 400 ppm hydrocarbons;
from about 50 to about 2500 ppm oxides of nitrogen; and,
from about 10 to about 150 ppm oxides of sulfur

16. The method of claim 13 wherein the combustion conditions are effective to produce diesel exhaust comprising:
from about 5 to about 1500 ppm carbon monoxide;
from about 20 to about 400 ppm hydrocarbons;
from about 50 to about 2500 ppm oxides of nitrogen; and,
from about 10 to about 150 ppm oxides of sulfur.

17. The method of claim 12 wherein the diesel exhaust comprises 0.1 g/mi or more unburned carbon particulate matter comprising adsorbed organic compounds selected from the group consisting of aldehydes, polycyclic aromatic hydrocarbons, and combinations thereof.

18. The method of claim 13 wherein the diesel exhaust comprises 0.1 g/mi or more unburned carbon particulate matter comprising adsorbed organic compounds selected from the group consisting of aldehydes, polycyclic aromatic hydrocarbons, and combinations thereof.

19. The method of claim 12 wherein the diesel exhaust comprises 0.1 g/mi or more unburned carbon particulate matter comprises adsorbed organic compounds selected from the group consisting of formaldehyde, acrolein, and combinations thereof.

20. The method of claim 13 wherein the diesel exhaust comprises 0.1 g/mi or more unburned carbon particulate matter comprises adsorbed organic compounds selected from the group consisting of formaldehyde, acrolein, and combinations thereof.

21. The method of claim 1 further comprising evaluating the contaminated component.

22. The method of claim 8 further comprising evaluating the contaminated diesel particulate filter.

23. The method of claim 20 further comprising evaluating the contaminated diesel particulate filter.

24. The method of claim 6 wherein the aging conditions comprise exposing the component to diesel exhaust at a flowrate of from about 0 to about 300 standard cubic feet per minute (scfm) at an exhaust temperature of from about 150° C. to about 650° C. for a period of time effective to age the component.

25. The method of claim 6 wherein the aging conditions comprise exposing the component to diesel exhaust at a flowrate of from about 0 to about 300 standard cubic feet per minute (scfm) at an exhaust temperature of from about 150° C. to about 300° C. for a period of time effective to age the component.

26. The method of claim 8 wherein the aging conditions comprise exposing the component to diesel exhaust at a flowrate of from about 0 to about 300 standard cubic feet per minute (scfm) at an exhaust temperature of from about 150° C. to about 650° C. for a period of time effective to age the component.

27. The method of claim 8 wherein the aging conditions comprise exposing the component to diesel exhaust at a flowrate of from about 0 to about 300 standard cubic feet per minute (scfm) at an exhaust temperature of from about 150° C. to about 300° C. for a period of time effective to age the component.

28. The method of claim 23 wherein the aging conditions comprise exposing the component to diesel exhaust at a flowrate of from about 0 to about 300 standard cubic feet per minute (scfm) at an exhaust temperature of from about 150° C. to about 650° C. for a period of time effective to age the component.

29. The method of claim 23 wherein the aging conditions comprise exposing the component to diesel exhaust at a flowrate of from about 0 to about 300 standard cubic feet per minute (scfm) at an exhaust temperature of from about 150° C. to about 300° C. for a period of time effective to age the component.

30. The method of claim 8 wherein the regeneration conditions comprise temperatures of from about 300° C. to about 650° C.

31. The method of claim 10 wherein the regeneration conditions comprise temperatures of from about 300° C. to about 650° C.

32. The method of claim 8 wherein the regeneration conditions comprise temperatures selected from the group consisting of about 350° C. or higher for catalyzed diesel particulate filters and about 600° C. or higher for uncatalyzed diesel particulate filters.

33. The method of claim 8 wherein the regeneration conditions are effective to produce diesel contaminant particulates having a minimum temperature of from about 500° C. to about 650° C.

34. The method of claim 8 wherein the regeneration conditions are effective to produce diesel contaminant particulates having a minimum temperature of from about 550° C. to about 650° C.

35. The method of claim 8 wherein the regeneration conditions are effective to produce diesel contaminant particulates having a minimum temperature of from about 585° C. to about 625° C.

36. The method of claim 30 wherein the regeneration conditions further comprise from about 3 vol. % to 20 vol. % oxygen in the exhaust stream.

37. The method of claim 33 wherein the regeneration conditions further comprise from about 3 vol. % to 20 vol. % oxygen in the exhaust stream.

38. The method of claim 34 wherein the regeneration conditions further comprise from about 3 vol. % to 20 vol. % oxygen in the exhaust stream.

39. The method of claim 35 wherein the regeneration conditions further comprise from about 3 vol. % to 20 vol. % oxygen in the exhaust stream.

40. The method of claim 36 wherein the regeneration conditions are maintained for about 20 minutes or less.

41. The method of claim 37 wherein the regeneration conditions are maintained for about 20 minutes or less.

42. The method of claim 39 wherein the regeneration conditions are maintained for about 20 minutes or less.

43. A method for aging a diesel particulate filter comprising:
providing a non-engine based test system comprising a combustor in fluid communication with the diesel particulate filter;
supplying diesel fuel and air to the combustor at a controlled air to fuel ratio (AFR) and under feed conditions effective to produce a feedstream flowpath effective to prevent substantial damage to the combustor;
combusting at least a portion of the diesel fuel in the feedstream flowpath under combustion conditions effective to produce diesel exhaust comprising one or more particulates; and exposing the component to the diesel exhaust under test conditions effective to produce a contaminated diesel particulate filter comprising contaminant particulates.

44. The method of claim 43 wherein the test conditions comprise aging conditions.

45. The method of claim 43 further comprising exposing the contaminated component to regeneration conditions effective to reduce the amount of diesel contaminant particulates, producing one or more regenerated diesel particulate filter.

46. The method of claim 44 further comprising exposing the contaminated component to regeneration conditions effective to reduce the amount of diesel contaminant particulates, producing one or more regenerated diesel particulate filters.

47. The method of claim 45 further comprising
providing a test diesel exhaust comprising a first quantity of particulate matter in grams per horsepower per hour (g/hp-hr); and,
passing the test diesel exhaust through one of the regenerated diesel particulate filters to produce a resulting final diesel exhaust comprising a second quantity of particulate matter which is about 50% or less (g/hp-hr) than the first quantity of particulate matter.

48. The method of claim 46 further comprising
providing a test diesel exhaust comprising a first quantity of particulate matter in grams per horsepower per hour (g/hp-hr); and,
passing the test diesel exhaust through one of the regenerated diesel particulate filters to produce a resulting final diesel exhaust comprising a second quantity of particulate matter which is from about 50% to about 90% (g/hp-hr) less than the first quantity of particulate matter.

49. The method of claim 43 wherein the combustion conditions are effective to produce diesel exhaust comprising exhaust components selected from the group consisting of carbon monoxide, carbon dioxide, oxides of nitrogen, oxides of sulfur, hydrocarbons, unburned carbon particulate matter, oxygen, nitrogen, and combinations thereof.

50. The method of claim 46 wherein the combustion conditions are effective to produce diesel exhaust comprising exhaust components selected from the group consisting of carbon monoxide, carbon dioxide, oxides of nitrogen, oxides of sulfur, hydrocarbons, unburned carbon particulate matter, oxygen, nitrogen, and combinations thereof.

51. The method of claim 47 wherein the combustion conditions are effective to produce diesel exhaust comprising exhaust components selected from the group consisting of carbon monoxide, carbon dioxide, oxides of nitrogen, oxides of sulfur, hydrocarbons, unburned carbon particulate matter, oxygen, nitrogen, and combinations thereof.

52. The method of claim 51 wherein the combustion conditions are effective to produce diesel exhaust further comprising exhaust components selected from the group consisting of phosphorous, zinc, aldehydes, nitrogen dioxide, sulfur dioxide.

53. The method of claim 50 wherein the combustion conditions are effective to produce diesel exhaust comprising:
from about 5 to about 1500 ppm carbon monoxide;
from about 20 to about 400 ppm hydrocarbons;
from about 50 to about 2500 ppm oxides of nitrogen; and,
from about 10 to about 150 ppm oxides of sulfur.

54. The method of claim 51 wherein the combustion conditions are effective to produce diesel exhaust comprising:
from about 5 to about 1500 ppm carbon monoxide;
from about 20 to about 400 ppm hydrocarbons;
from about 50 to about 2500 ppm oxides of nitrogen; and,
from about 10 to about 150 ppm oxides of sulfur.

55. The method of claim 49 wherein the diesel exhaust comprises 0.1 g/mi or more unburned carbon particulate matter comprising adsorbed organic compounds selected from the group consisting of aldehydes, polycyclic aromatic hydrocarbons, and combinations thereof.

56. The method of claim 51 wherein the diesel exhaust comprises 0.1 g/mi or more unburned carbon particulate matter comprising adsorbed organic compounds selected from the group consisting of aldehydes, polycyclic aromatic hydrocarbons, and combinations thereof.

57. The method of claim 49 wherein the diesel exhaust comprises 0.1 g/mi or more unburned carbon particulate matter comprises adsorbed organic compounds selected from the group consisting of formaldehyde, acrolein, and combinations thereof.

58. The method of claim 51 wherein the diesel exhaust comprises 0.1 g/mi or more unburned carbon particulate matter comprises adsorbed organic compounds selected from the group consisting of formaldehyde, acrolein, and combinations thereof.

59. The method of claim 43 further comprising evaluating the contaminated component.

60. The method of claim 47 further comprising evaluating the contaminated diesel particulate filter.

61. The method of claim 58 further comprising evaluating the contaminated diesel particulate filter.

62. The method of claim 44 wherein the aging conditions comprise exposing the component to diesel exhaust at a flowrate of from about 0 to about 300 standard cubic feet per minute (scfm) at an exhaust temperature of from about 150° C. to about 650° C. for a period of time effective to age the component.

63. The method of claim 44 wherein the aging conditions comprise exposing the component to diesel exhaust at a flowrate of from about 0 to about 300 standard cubic feet per minute (scfm) at an exhaust temperature of from about 150° C. to about 300° C. for a period of time effective to age the component.

64. The method of claim 47 wherein the aging conditions comprise exposing the component to diesel exhaust at a flowrate of from about 0 to about 300 standard cubic feet per minute (scfm) at an exhaust temperature of from about 150° C. to about 650° C. for a period of time effective to age the component.

65. The method of claim 47 wherein the aging conditions comprise exposing the component to diesel exhaust at a flowrate of from about 0 to about 300 standard cubic feet per minute (scfm) at an exhaust temperature of from about 150° C. to about 300° C. for a period of time effective to age the component.

66. The method of claim 62 wherein the regeneration conditions comprise temperatures of from about 300° C. to about 650° C.

67. The method of claim 64 wherein the regeneration conditions comprise temperatures of from about 300° C. to about 650° C.

68. The method of claim 64 wherein the regeneration conditions comprise temperatures are selected from the group of about 350° C. or higher for catalyzed diesel particulate filters and about 600° C. or higher for uncatalyzed diesel particulate filters.

69. The method of claim 64 wherein the regeneration conditions are effective to produce diesel contaminant particulates having a minimum temperature of from about 500° C. to about 650° C.

70. The method of claim 64 wherein the regeneration conditions are effective to produce diesel contaminant particulates having a minimum temperature of from about 550° C. to about 650° C.

71. The method of claim 64 wherein the regeneration conditions are effective to produce diesel contaminant particulates having a minimum temperature of from about 585° C. to about 625° C.

72. The method of claim 62 wherein the regeneration conditions further comprise from about 3 vol. % to 20 vol. % oxygen in the exhaust stream.

73. The method of claim 64 wherein the regeneration conditions further comprise from about 3 vol. % to 20 vol. % oxygen in the exhaust stream.

74. The method of claim 69 wherein the regeneration conditions further comprise from about 3 vol. % to 20 vol. % oxygen in the exhaust stream.

75. The method of claim 72 wherein the regeneration conditions are maintained for about 20 minutes or less.

76. The method of claim 73 wherein the regeneration conditions are maintained for about 20 minutes or less.

77. The method of claim 74 wherein the regeneration conditions are maintained for about 20 minutes or less.

78. A method for regenerating a particulate contaminated component comprising:
providing a particulate contaminated component;
providing a combustor in fluid communication with the particulate contaminated component comprising contaminant particulates;
supplying fuel and air to the combustor at a controlled air to fuel ratio (AFR) and under feed conditions effective to combust at least a portion of the fuel and to produce a feedstream flowpath effective to prevent substantial damage to the combustor;
exposing the particulate contaminated component to the exhaust under regeneration conditions effective to reduce the amount of contaminant particulates.

79. A method for regenerating a particulate contaminated diesel particulate filter (DPF) comprising:
exposing a DPF to diesel exhaust under conditions effective to produce a particulate contaminated DPF comprising an amount of contaminant particulates;
providing a combustor in fluid communication with the contaminated DPF;
supplying fuel and air to the combustor at a controlled air to fuel ratio (AFR) and under feed conditions effective to combust at least a portion of the fuel and to produce a feedstream flowpath comprising exhaust;
exposing the contaminated DPF to regeneration conditions effective to reduce the amount of contaminant particulates and to produce one or more regenerated DPFs.

80. The method of claim 79 wherein the regeneration conditions comprise diesel exhaust.

81. The method of claim 80 further comprising
providing a test diesel exhaust comprising a first quantity of particulate matter in grams per horsepower per hour (g/hp-hr); and,
passing the test diesel exhaust through one of the regenerated diesel particulate filters to produce a resulting final diesel exhaust comprising a second quantity of particulate matter which is about 50% or less (g/hp-hr) than the first quantity of particulate matter.

82. The method of claim 80 further comprising
providing a test diesel exhaust comprising a first quantity of particulate matter in grams per horsepower per hour (g/hp-hr); and,
passing the test diesel exhaust through one of the regenerated diesel particulate filters to produce a resulting final diesel exhaust comprising a second quantity of particulate matter which is from about 50% to about 90% (glhp-hr) less than the first quantity of particulate matter.

83. The method of claim 80 wherein the regeneration conditions comprise temperatures of from about 300° C. to about 650° C.

84. The method of claim 81 wherein the regeneration conditions comprise temperatures of from about 300° C. to about 650° C.

85. The method of claim 81 wherein the regeneration conditions comprise temperatures are selected from the group of about 350° C. or higher for catalyzed diesel particulate filters and about 600° C. or higher for uncatalyzed diesel particulate filters.

86. The method of claim 81 wherein the regeneration conditions are effective to produce diesel contaminant particulates having a minimum temperature of from about 500° C. to about 650° C.

87. The method of claim 81 wherein the regeneration conditions are effective to produce diesel contaminant particulates having a minimum temperature of from about 550° C. to about 650° C.

88. The method of claim 81 wherein the regeneration conditions are effective to produce diesel contaminant particulates having a minimum temperature of from about 585° C. to about 625° C.

89. The method of claim 83 wherein the regeneration conditions further comprise from about 3 vol. % to 20 vol. % oxygen in the exhaust stream.

90. The method of claim 86 wherein the regeneration conditions further comprise from about 3 vol. % to 20 vol. % oxygen in the exhaust stream.

91. The method of claim 87 wherein the regeneration conditions further comprise from about 3 vol. % to 20 vol. % oxygen in the exhaust stream.

92. The method of claim 88 wherein the regeneration conditions further comprise from about 3 vol. % to 20 vol. % oxygen in the exhaust stream.

93. The method of claim 89 wherein the regeneration conditions are maintained for about 20 minutes or less.

94. The method of claim 90 wherein the regeneration conditions are maintained for about 20 minutes or less.

95. The method of claim 92 wherein the regeneration conditions are maintained for about 20 minutes or less.

96. The method of claim 95 wherein the feedstream flowpath is effective to prevent flame from remaining in constant contact with an inner wall of the combustor during the combusting.
